(19) United States
(12) Patent Application Publication
(10) Pub. No.: US 2026/0275291 A1
(43) Pub. Date: Sep. 17, 2026

(54) ALLOGENEIC AND XENOGENEIC SUBCUTANEOUS ADIPOSE TISSUE AND METHODS FOR PRESERVING SAME

(71) Applicant: SinUse Corp., West Hollywood, CA (US)

(72) Inventors: Gabriel Zada, Los Angeles, CA (US); Seyed Mohammadreza Ghodsi, West Hollywood, CA (US)

(21) Appl. No.: 19/470,746

(22) PCT Filed: Mar. 29, 2024
(86) PCT No.: PCT/US2024/022401

§ 371 Date: Sep. 29, 2025

Related U.S. Application Data
(60) Provisional application No. 63/493,667, filed on Mar. 31, 2023.

(51) Int. Cl. C12N 5/077; A61L 27/36

(52) U.S. Cl. CPC C12N 5/0653; A61L 27/3604; A61L 27/3683

(57) ABSTRACT

Disclosed herein are embodiments of a processed allogeneic and xenogeneic subcutaneous adipose tissue for reconstruction and cosmetic surgeries and method for preparing allogeneic and xenogeneic subcutaneous adipose tissue for reconstruction and cosmetic surgeries. The preservation method can include the subcutaneous adipose tissue being subjected to a set of physiochemical treatments to prohibit host immunogenic response upon transplantation/implantation.

### CROSS-REFERENCE TO RELATED APPLICATIONS

This application claims priority to U.S. Provisional Appl. No. 63/493,667, filed on Mar. 31, 2023, which is hereby incorporated by reference in its entirety.

### FIELD OF THE DISCLOSURE

This disclosure relates to processed allogeneic and xenogeneic subcutaneous adipose tissue for reconstruction and cosmetic surgeries and methods for preparing allogeneic and xenogeneic subcutaneous adipose tissue for reconstruction and cosmetic surgeries. Embodiments and examples disclosed herein may allow for the implantation of allogeneic and xenogeneic subcutaneous adipose tissue without evoking adverse host immunogenic response.

### BACKGROUND

Autologous adipose or fat grafting is a common surgical procedure performed daily in the U.S. for purposes of harvesting this material for reconstruction. Autologous fat grafting has been in use for decades, where fat tissue is harvested during an operation from the patient (e.g., from the abdomen, thigh, or another source) and used to reconstruct, repair, or obliterate dead space in other anatomical regions being operated. For instance, autologous fat grafting and repair is utilized frequently in head and neck surgery, sinus surgery, neurosurgery, skull base surgery, plastic surgery, and cosmetic surgery procedures, among others. For example, obliteration of the frontal sinus is an important procedure that has critical benefits to many patients worldwide, including preventing or reducing the likelihood of chronic infections, obliterating cerebrospinal fluid fistulas, addressing head trauma issues, and/or treating cancer in some patients. Filler materials are often used to obliterate dead space in sinuses. For example, surgeons conventionally harvest fat (e.g., abdominal fat grafts) from the patient for use in sinus obliteration procedures. In fact, harvested fat has become the most commonly used obliteration material for fat grafting procedures, including the frontal sinus. Fat grafting is widely used during pituitary operations and both open and endoscopic skull base surgery operations. Fat grafting is widely used during pharyngeal reconstruction operations.

However, using harvested fat from the patient all too commonly introduces significant complications and drawbacks for the patient, which often become more burdensome and threatening to the patient than any impacts on or around the sinuses, which is the primary surgical location. For example, harvesting the fat from the patient results in an additional surgical site on the patient's body that must be closed, often by subcutaneous and skin sutures. This results in additional operating room and anesthesia time, additional pain and discomfort, and additional risk for complications such as hematoma and infection. Additionally, the wound site must be cared for after the procedure to ensure that infection and other injury to the patient is avoided. The graft site is often more likely to become infected than the sinus or skull base location. Additionally, the graft location can be very painful and debilitating for the patient, and the healing process can be much longer for the graft location than the sinus location, which is the primary surgical location, causing patients to utilize more opiates or pain medicines. Finally, grafting commonly results in permanent scarring or other skin defects for the patient and other suboptimal cosmetic outcomes. Additional cost of fat grafting is passed to patients, payers, insurers, hospital systems, and ultimately society and taxpayers.

Embodiments and examples disclosed herein may provide acceptable alternative filler materials to avoid the complications and drawbacks from conventional autologous fat grafting procedures.

### SUMMARY OF SOME EXEMPLIFYING EMBODIMENTS

The methods of this disclosure each have several innovative aspects, implementations, or aspects, no single one of which is solely responsible for the desirable attributes disclosed herein.

Disclosed here are embodiments of allogeneic and xenogeneic subcutaneous adipose tissue for reconstruction and cosmetic surgeries and methods of harvesting, processing, producing, and/or preserving allogeneic and xenogeneic subcutaneous adipose tissue for reconstruction and cosmetic surgeries.

In some embodiments, the tissue product/implant includes an adipose tissue extracted from a human, cadaveric or animal (e.g., bovine or porcine or ovine) donor/cadaver. The adipose tissue can be processed and can contain collagen, elastin, fibrin, lipids and extracellular matrix, or any combination of the foregoing. In some embodiments, the tissue product/implant is allogeneic. In some embodiments, the tissue product/implant is xenogeneic. The adipose tissue can be stored with a stable shelf life and subsequently configured for use in cosmetic and reconstruction surgeries. The adipose tissue is used for cosmetic and reconstruction surgeries. The adipose tissue can be processed or configured to be free of all immunogenic factors so it is compatible with any recipient patient.

The adipose tissue can be stored in a concentrated ionic solution to remove the polar cellular components. In some embodiments, the concentrated ionic solution can further contain a lipase inhibitor. In some embodiments, the adipose tissue is malleable and trimmable. In some embodiments, the adipose tissue is fresh. In some embodiments, the adipose tissue is lyophilized in a mixture of sucrose and dextran solution. In some embodiments, the adipose tissue is sterilized using gamma radiation, ultraviolet radiation, or ozone exposure. In some embodiments, the adipose tissue is stored in a vacuum-sealed plastic container for up to 12 months.

Also disclosed herein are embodiments of a method of making a tissue product that can include an intact adipose tissue that is harvested from a donor/cadaver. In some embodiments, the tissue product/implant is allogeneic. In some embodiments, the tissue product/implant is xenogeneic. The subcutaneous adipose tissue can be subjected to a set of physiochemical treatments to extract all, or substantially all (e.g., over 85%, over 90%, or over 95%) of the immunogenic factors. The physiochemical treatments can include receiving an adipose tissue, trimming vasculatures, skin, and fascia from the adipose tissue; incubating the adipose tissue in a buffer solution until freezing; thawing the frozen adipose tissue using a cryogenic solution; incubating the adipose tissue in a digestion solution; incubating the adipose tissue in a concentrated ionic solution overnight; extracting the polar cellular components from the adipose tissue; incubating the subcutaneous adipose tissue in a non-ionic or ionic surfactant solutions; and washing the subcutaneous adipose tissue in ethanol or IPA or methanol.

In some embodiments, the buffer solution can include 25 mM HEPES. In some embodiments, the cryogenic solution comprises of 1% lipase inhibitor. In some embodiments, the digestion solution comprises of 0.25% EDTA-trypsin solution. In some embodiments, the adipose tissue is incubated in the digestion solution between 6-24 hours. In some embodiments, the concentrated ionic solution comprises of 1M NaCl. In some embodiments, the concentrated ionic solution further comprises of 1% lipase inhibitor. In some embodiments, adipose tissue is incubated in concentrated ionic solution between 1-3 hours. In some embodiments, the non-ionic solution comprises of 0.5% Triton X-100. In some embodiments, the adipose tissue was further incubated in antibacterial and antifungal solutions. In some embodiments, the adipose tissue is free of proteins, sugars, amino acids, DNAs, RNAs, and nucleic acids. In some embodiments, the adipose tissue is free of all immunogenic components. In some embodiments, the adipose tissue is used for cosmetic and reconstruction surgeries. In some embodiments, the adipose tissue is malleable and trimmable.

In certain embodiments, a method of preserving subcutaneous adipose tissue for cosmetic and reconstruction surgeries may include receiving an adipose tissue from a donor/cadaver, trimming vasculatures, skin, and fascia from the adipose tissue; incubating the adipose tissue in a buffer solution until freezing; thawing the frozen adipose tissue in cryogenic solution; incubating the adipose tissue in a digestion solution; incubating the adipose tissue in a concentrated ionic solution; extracting the polar cellular components from the adipose tissue; incubating the subcutaneous adipose tissue in a non-ionic or ionic surfactant solutions; washing the subcutaneous adipose tissue in ethanol, IPA, methanol; wherein the adipose tissue is processed; lyophilizing the adipose tissue, sterilizing the processed adipose tissue; and incubating the adipose tissue in an anti-inflammatory/antibiotics cocktail solution and in a mixture of sucrose and dextran solution; packaging the sterilized adipose tissue in a vacuum-sealed plastic container; and storing the sterilized adipose tissue. In some embodiments, the preservation steps further includes lyophilization of the processed adipose tissue. In some embodiments, the buffer solution comprises of 25 mM HEPES. In some embodiments, the cryogenic solution comprises of 1% lipase inhibitor. In some embodiments, the digestion solution comprises of 0.25% EDTA-trypsin solution. In some embodiments, the adipose tissue is incubated in the digestion solution between 6-24 hours. In some embodiments, the concentrated ionic solution comprises of 1M NaCl. In some embodiments, the concentrated ionic solution further comprises of 1% lipase inhibitor. In some embodiments, the adipose tissue is incubated in the concentrated ionic solution between 1-3 hours. In some embodiments, the non-ionic solution comprises of 0.5% Triton X-100. In some embodiments, the sterilization of the adipose tissue comprises irradiation from a group selected from Gamma radiation, ultraviolet radiation, or ozone exposure. In some embodiments, the adipose tissue was further incubated in antibacterial and antifungal solutions. In some embodiments, the adipose tissue is free of proteins, sugars, amino acids, DNAs, RNAs, and nucleic acids. In some embodiments, the adipose tissue is free of all immunogenic components. In some embodiments, the preservation step further comprises storing the sterilized adipose tissue at 4° C. for up to 6 months. In some embodiments, the preservation step further comprises storing the sterilized adipose tissue at −20° C. for up to 12 months. In some embodiments, the sterilized adipose tissue is reconstituted in a saline solution and stored at −20° C. for up to 12 months. In some embodiments, the sterilized adipose tissue is reconstituted in a ringer solution and stored at −20° C. for up to 12 months. In some embodiments, the adipose tissue is fresh. In some embodiments, the adipose tissue is malleable and trimmable.

### BRIEF DESCRIPTION OF THE DRAWINGS

A better understanding of the features and advantages of the present disclosure will be obtained by reference to the detailed description below that sets forth illustrative embodiments, in which the principles disclosed herein are utilized, and the accompanying drawings of which:

FIG. 1 illustrates a flow diagram of one method of performing a method of preserving allogeneic and xenogeneic subcutaneous adipose tissue for reconstruction and cosmetic surgeries according to embodiments of the present disclosure.

FIG. 2A-D illustrate photomicrographs of the collagen fiber of a bovine adipose tissue.

FIG. 3A-B illustrate photomicrographs of the collagen fiber of a non-processed bovine adipose tissue.

### DETAILED DESCRIPTION OF SOME EMBODIMENTS

Disclosed herein are examples and embodiments of tissue prepared for fat grafting procedures that have a significant advantage to the patient as compared to tissue harvested from the patient's body. By producing biocompatible tissue/material for sinus or other surgical/anatomical space obliteration, the surgeon can perform the repair or obliteration procedure without the need to surgically harvest tissue from the patient during the index procedure.

By eliminating a major step in conventional fat grafting surgical procedures (i.e., by eliminating the need to harvest tissue from the patient during the procedure), the tissue embodiments and the surgical method embodiments disclosed herein can have the advantage(s) of significantly reducing the total time of the overall surgical procedure. This can result in a significantly improved level of care for the patient and a significant savings for the cost of the procedure, anesthesia time and side effects, resulting in increased efficiencies and cost savings for patients, hospitals, and others in the medical industry. Similarly, eliminating the additional incision and harvesting of the patient also improves the level of care to the patient and results in additional cost savings by eliminating all of the post-surgical care that is required to treat the graft wound site during the post-surgical healing phase and eliminates the risk of infection to the patient at the graft location. Pain and discomfort is improved with reduced need for pain medicine and the potential for earlier ambulation and mobility.

Embodiments of a method of preparing and preserving allogeneic and xenogeneic subcutaneous adipose tissue for reconstruction and cosmetic surgeries are disclosed herein. Any embodiments of the adipose tissue disclosed herein can be configured to be soft and malleable and/or otherwise optimized for use as a filler for sinus or other anatomical compartment ablation procedures.

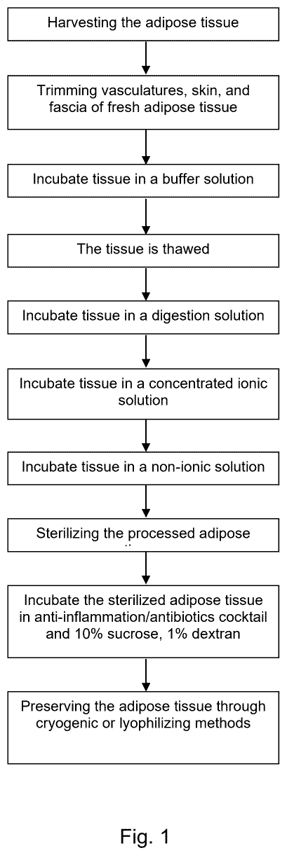

FIG. 1 is a flow diagram of an overview of an example of a method of preserving allogeneic and xenogeneic subcutaneous adipose tissue for reconstruction and cosmetic surgeries. The flow diagram exhibits that the method begins with harvesting the adipose tissue from a donor/cadaver followed by tramming the vasculatures, skin and fascia of the fresh adipose tissue. Then the adipose tissue is processed by incubating the adipose tissue in a buffer solution until frozen. Then the frozen tissue is thawed, incubated in a digestion solution and then incubated in a concentrated ionic solution. Then the adipose tissue was incubated in a non-ionic solution. The processed adipose tissue is then sterilized. The sterilized adipose tissue is then incubated in an anti-inflammatory/antibiotics cocktail and sucrose and dextran solution. Then the sterilized tissue is preserved through cryogenic method or lyophilization.

### Subcutaneous Adipose Tissue

In some embodiments, a subcutaneous adipose tissue comprising an adipose tissue is extracted from a donor/cadaver. In some embodiments, the adipose tissue can be allogeneic. In some embodiments, the adipose tissue can be xenogeneic. The extracted adipose tissue can be fresh. The extracted adipose tissue can then be processed and can contain collagen, fibrin, elastin, lipids and extracellular matrix. The processed adipose tissue can be sterilized using irradiation from a group selected from gamma radiation, ultraviolet radiation, or ozone exposure.

Some embodiments of the subcutaneous adipose tissue can be malleable and configured for use in reconstruction surgeries, including cosmetic surgeries. Some embodiments of the subcutaneous adipose tissue can be processed and otherwise configured to be free of all immunogenic factors. The subcutaneous adipose tissue can be processed and otherwise configured to be free of proteins, sugars, amino acids, DNAs, RNAs, and nucleic acids.

Some embodiments of the subcutaneous adipose tissue can be packaged in a vacuum-sealed plastic container and stored for up to 12 months. Some embodiments of the subcutaneous adipose tissue can also be stored in a saline or ringer solution and stored for up to 12 months. Some embodiments of the subcutaneous adipose tissue can be reconstituted in a saline or ringer solution before use.

### Methods of Processing

In some embodiments, a method of making an allogeneic subcutaneous adipose tissue disclosed herein can include application of a set of treatment to an intact adipose tissue from a donor/cadaver of the same species that may include receiving an adipose tissue, trimming vasculatures, skin, and fascia from the adipose tissue, incubating the adipose tissue in a buffer solution in freezing temperature, thawing the frozen adipose tissue using a cryogenic solution, incubating the adipose tissue in a digestion solution, incubating the adipose tissue in a concentrated ionic solution, extracting the polar cellular components from the adipose tissue, incubating the subcutaneous adipose tissue in a non-ionic surfactant solutions, and washing the subcutaneous adipose tissue in ethanol or IPA or methanol. Some embodiments of the method can include any combination of any of the foregoing.

In some embodiments, the adipose tissue extracted from donor/cadaver can be processed as a fresh tissue. In some embodiments, the adipose tissue is incubated in the buffer solution until freezing, which can disrupt the phospholipid membrane and to expose the cytoplasm and nucleus. In some embodiments, the adipose tissue can be frozen at a temperature of −80.0° C. or approximately −80.0° C., or ranging from −75.0° C. or about −75.0° C. to −85.0° C. or about −85.0° C., such as −75.0° C., −76.0° C., −77.0° C., −78.0° C., −79.0° C., −80.0° C., −81.0° C., −82.0° C., −83.0° C., −84.0° C., −85.0° C., or ranges including and/or spanning the aforementioned values. In some embodiments, the buffer solution can include 25 mM HEPES. In some embodiments, the frozen adipose tissue is thawed in a cryogenic solution. In some embodiments, the frozen adipose tissue is thawed at a temperature ranging from about 36.0° C. to about 38.0° C., such as 36.0° C., 36.5° C., 37.0° C., 37.5° C., 38.0° C., or ranges including and/or spanning the aforementioned values. In some embodiments, the cryogenic solution can contain 1% or approximately 1% of a lipase inhibitor. The cryogenic solution can include an antibacterial and antifungal cocktail solution. In some embodiments, the lipase inhibitor can be Tetrahydrolipstatin. In some embodiments, the adipose tissue is incubated in a digestion solution with agitation at a temperature ranging from about 36.0° C. to about 38.0° C., such as 36.0° C., 36.5° C., 37.0° C., 37.5° C., 38.0° C., or ranges including and/or spanning the aforementioned values. In some embodiments, the adipose tissue is incubated in a digestion solution for a time ranging from about 6 hours to about 24 hours, such as 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or ranges including and/or spanning the aforementioned values. The digestion solution can contain EDTA-Trypsin solution at a concentration of 0.25% or about 0.25%, or ranging from about 0.15% to about 0.35%, such as 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, or ranges including and/or spanning the aforementioned values. In some embodiments, the digestion solution can include an antibacterial and antifungal cocktail solution.

In some embodiments, the adipose tissue is incubated in a concentrated ionic solution at a temperature ranging from about 36.0° C. to about 38.0° C., such as 36.0° C., 36.5° C., 37.0° C., 37.5° C., 38.0° C., or ranges including and/or spanning the aforementioned values. In some embodiments, the concentrated ionic solution contains antibacterial and antifungal cocktail solution. In some embodiments, the adipose tissue is incubated in a concentrated ionic solution for a time ranging from about 1 hours to about 3 hours, such as 1 hours, 1.5 hours, 2 hours, 2.5 hours, 3 hours, or ranges including and/or spanning the aforementioned values. In some embodiments, the concentrated ionic solution can contain NaCl to extract the polar cellular component at a concentration ranging from about 0.5M to about 2M, such as 0.5M, 0.6M, 0.7M, 0.8M, 0.9M, 1.0M, 1.1M, 1.2M, 1.3M, 1.4M, 1.5M, 1.6M, 1.7M, 1.8M, 1.9M, 2.0M, or ranges including and/or spanning the aforementioned values. The concentrated ionic solution can contain 1% or approximately 1% of a lipase inhibitor.

In some embodiments, the adipose tissue is incubated in a non-ionic surfactant to extract all the cellular component. The non-ionic surfactant can contain Triton X-100 solution at a concentration ranging from about 0.1% to about 1.0%, such as 0.1%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.0. %, or ranges including and/or spanning the aforementioned values. The adipose tissue can be incubated in the non-ionic surfactant for a time ranging from about 1 hours to about 3 hours, such as 1 hours, 1.5 hours, 2 hours, 2.5 hours, 3 hours, or ranges including and/or spanning the aforementioned values.

In some embodiments, the adipose tissue is washed in ethanol or IPA or methanol or IPA or methanol with agitation for a time ranging from about 1 hours to about 3 hours, such as 1 hours, 1.5 hours, 2 hours, 2.5 hours, 3 hours, or ranges including and/or spanning the aforementioned values. In some embodiments, the adipose tissue is washed in ethanol or IPA or methanol at a temperature ranging from about 2.0° C. to about 6.0° C., such as 2.0° C., 2.5° C., 3.0° C., 3.5° C., 4.0° C., 4.5° C., 5.0° C., 5.5° C., 6.0° C., or ranges including and/or spanning the aforementioned values.

In certain embodiments, a method of making a xenogeneic subcutaneous adipose tissue includes application of a set of treatments to an intact adipose tissue from a donor/cadaver of a different species from the species receiving the adipose tissue, trimming vasculatures, skin, and fascia from the adipose tissue; incubating the adipose tissue in a buffer solution until freezing; thawing the frozen adipose tissue using a cryogenic solution; incubating the adipose tissue in a digestion solution; incubating the adipose tissue in a concentrated ionic solution; extracting polar cellular components from the adipose tissue; incubating the subcutaneous adipose tissue in a non-ionic surfactant solutions; and washing the subcutaneous adipose tissue in ethanol or IPA or methanol.

In some embodiments, the adipose tissue extracted from the donor/cadaver can be processed as a fresh tissue. In some embodiments, the adipose tissue is incubated in the buffer solution until freezing which can disrupt the phospholipid membrane and expose the cytoplasm and nucleus. In some embodiments, the adipose tissue can be frozen at a temperature ranging from about −75.0° C. to about −85.0° C., such as −75.0° C., −76.0° C., −77.0° C., −78.0° C., −79.0° C., −80.0° C., −81.0° C., −82.0° C., −83.0° C., −84.0° C., −85.0° C., or ranges including and/or spanning the aforementioned values. In some embodiments, the buffer solution contains 25 mM HEPES. In some embodiments, the frozen adipose tissue is thawed in a cryogenic solution. In some embodiments, the frozen adipose tissue is thawed at a temperature ranging from about 36.0° C. to about 38.0° C., such as 36.0° C., 36.5° C., 37.0° C., 37.5° C., 38.0° C., or ranges including and/or spanning the aforementioned values. In some embodiments, the cryogenic solution can contain 1% or approximately 1% of a lipase inhibitor. The cryogenic solution can include an antibacterial and antifungal cocktail solution. In some embodiments, the lipase inhibitor can be Tetrahydrolipstatin. In some embodiments, the adipose tissue is incubated in a digestion solution with agitation at a temperature ranging from about 36.0° C. to about 38.0° C., such as 36.0° C., 36.5° C., 37.0° C., 37.5° C., 38.0° C., or ranges including and/or spanning the aforementioned values. In some embodiments, the adipose tissue is incubated in a digestion solution for a time ranging from about 6 hours to about 24 hours, such as 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or ranges including and/or spanning the aforementioned values. The digestion solution can contain EDTA-Trypsin solution at a concentration ranging from about 0.15% to about 0.35%, such as 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, or ranges including and/or spanning the aforementioned values. In some embodiments, the digestion solution can include an antibacterial and antifungal cocktail solution.

In some embodiments, the adipose tissue is incubated in a concentrated ionic solution at a temperature ranging from about 36.0° C. to about 38.0° C., such as 36.0° C., 36.5° C., 37.0° C., 37.5° C., 38.0° C., or ranges including and/or spanning the aforementioned values. In some embodiments, the concentrated ionic solution contains antibacterial and antifungal cocktail solution. In some embodiments, the adipose tissue is incubated in a concentrated ionic solution for a time ranging from about 1 hours to about 3 hours, such as 1 hours, 1.5 hours, 2 hours, 2.5 hours, 3 hours, or ranges including and/or spanning the aforementioned values. In some embodiments, the concentrated ionic solution contains NaCl to extract the polar cellular component at a concentration ranging from about 0.5M to about 2M, such as 0.5M, 0.6M, 0.7M, 0.8M, 0.9M, 1.0M, 1.1M, 1.2M, 1.3M, 1.4M, 1.5M, 1.6M, 1.7M, 1.8M, 1.9M, 2.0M, or ranges including and/or spanning the aforementioned values. The concentrated ionic solution can contain 1% or approximately 1% of a lipase inhibitor.

In some embodiments, the adipose tissue is incubated in a non-ionic surfactant to extract all the cellular component. The non-ionic surfactant can contain Triton X-100 solution at a concentration ranging from about 0.1% to about 1.0%, such as 0.1%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.0. %, or ranges including and/or spanning the aforementioned values. The adipose tissue can incubated in the non-ionic surfactant for a time ranging from about 1 hours to about 3 hours, such as 1 hours, 1.5 hours, 2 hours, 2.5 hours, 3 hours, or ranges including and/or spanning the aforementioned values.

In some embodiments, the adipose tissue is washed in ethanol or IPA or methanol with agitation for a time ranging from about 1 hours to about 3 hours, such as 1 hours, 1.5 hours, 2 hours, 2.5 hours, 3 hours, or ranges including and/or spanning the aforementioned values. In some embodiments, the adipose tissue is washed in ethanol or IPA or methanol at a temperature ranging from about 2.0° C. to about 6.0° C., such as 2.0° C., 2.5° C., 3.0° C., 3.5° C., 4.0° C., 4.5° C., 5.0° C., 5.5° C., 6.0° C., or ranges including and/or spanning the aforementioned values.

### Methods of Preservation

In some embodiments, a method of preserving allogeneic subcutaneous adipose tissue disclosed herein can include application of a set of treatments to an intact adipose tissue from a donor/cadaver of the same species that may include receiving an adipose tissue, trimming vasculatures, skin, and fascia from the adipose tissue; incubating the adipose tissue in a buffer solution until freezing; thawing the frozen adipose tissue using a cryogenic solution; incubating the adipose tissue in a digestion solution; incubating the adipose tissue in a concentrated ionic solution; extracting polar cellular components from the adipose tissue; incubating the subcutaneous adipose tissue in a non-ionic surfactant solutions; washing the subcutaneous adipose tissue in ethanol or IPA or methanol; wherein the adipose tissue is processed; lyophilization of the adipose tissue, sterilizing the adipose tissue; incubating the adipose tissue in an anti-inflammatory/antibiotics cocktail and sucrose and dextran solution; storing the adipose tissue in a vacuum-sealed plastic container; and/or reconstituting the adipose tissue in a saline or a ringer solution before use. Any embodiments can include any combination of any of the foregoing steps or processes.

In some embodiments, the adipose tissue extracted from donor/cadaver of the same species. In some embodiments, the adipose tissue extracted from donor/cadaver can be processed as a fresh tissue. The adipose tissue can be incubated in the buffer solution until freezing which can disrupt the phospholipid membrane and to expose the cytoplasm and nucleus. In some embodiments, the adipose tissue can be frozen at a temperature ranging from about −75.0° C. to about −85.0° C., such as −75.0° C., −76.0° C., −77.0° C., −78.0° C., −79.0° C., −80.0° C., −81.0° C., −82.0° C., −83.0° C., −84.0° C., −85.0° C., or ranges including and/or spanning the aforementioned values. The buffer solution can contain 25 mM HEPES. In some embodiments, the frozen adipose tissue can be thawed in a cryogenic solution. In some embodiments, the frozen adipose tissue is thawed at a temperature ranging from about 36° C. to about 38° C., such as 36° C., 36.5° C., 37° C., 37.5° C., 38° C., or ranges including and/or spanning the aforementioned values. In some embodiments, the cryogenic solution can contain 1% or approximately 1% of a lipase inhibitor. The cryogenic solution can include an antibacterial and antifungal cocktail solution. In some embodiments, the lipase inhibitor can be Tetrahydrolipstatin. The adipose tissue can be incubated in a digestion solution with agitation at a temperature ranging from about 36.0° C. to about 38.0° C., such as 36.0° C., 36.5° C., 37.0° C., 37.5° C., 38.0° C., or ranges including and/or spanning the aforementioned values. In some embodiments, the adipose tissue is incubated in a digestion solution for a time ranging from about 6 hours to about 24 hours, such as 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or ranges including and/or spanning the aforementioned values. The digestion solution can contain EDTA-Trypsin solution at a concentration ranging from about 0.15% to about 0.35%, such as 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, or ranges including and/or spanning the aforementioned values. Any embodiments of the digestion solution can include an antibacterial and antifungal cocktail solution.

In some embodiments, the adipose tissue can be incubated in a concentrated ionic solution at a temperature ranging from about 36.0° C. to about 38.0° C., such as 36.0° C., 36.5° C., 37.0° C., 37.5° C., 38.0° C., or ranges including and/or spanning the aforementioned values. In some embodiments, the concentrated ionic solution contains antibacterial and antifungal cocktail solution. In some embodiments, the adipose tissue is incubated in a concentrated ionic solution for a time ranging from about 1 hours to about 3 hours, such as 1 hours, 1.5 hours, 2 hours, 2.5 hours, 3 hours, or ranges including and/or spanning the aforementioned values. In some embodiments, the concentrated ionic solution contains NaCl to extract the polar cellular component at a concentration ranging from about 0.5M to about 2M, such as 0.5M, 0.6M, 0.7M, 0.8M, 0.9M, 1.0M, 1.1M, 1.2M, 1.3M, 1.4M, 1.5M, 1.6M, 1.7M, 1.8M, 1.9M, 2.0M, or ranges including and/or spanning the aforementioned values. The concentrated ionic solution can contain 1% or approximately 1% of a lipase inhibitor.

The adipose tissue can be incubated in a non-ionic surfactant to extract all the cellular component. The non-ionic surfactant can contain Triton X-100 solution at a concentration ranging from about 0.1% to about 1.0%, such as 0.1%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.0. %, or ranges including and/or spanning the aforementioned values. In some embodiments, the adipose tissue is incubated in the non-ionic surfactant for a time ranging from about 1 hours to about 3 hours, such as 1 hours, 1.5 hours, 2 hours, 2.5 hours, 3 hours, or ranges including and/or spanning the aforementioned values.

In some embodiments, the adipose tissue is washed in ethanol or IPA or methanol with agitation for a time ranging from about 1 hours to about 3 hours, such as 1 hours, 1.5 hours, 2 hours, 2.5 hours, 3 hours, or ranges including and/or spanning the aforementioned values. In some embodiments, the adipose tissue can be washed in ethanol or IPA or methanol at a temperature ranging from about 2.0° C. to about 6.0° C., such as 2.0° C., 2.5° C., 3.0° C., 3.5° C., 4.0° C., 4.5° C., 5.0° C., 5.5° C., 6.0° C., or ranges including and/or spanning the aforementioned values.

In embodiments, the processed adipose tissue can be used fresh. The fresh processed adipose tissue can be sterilized using irradiation from a group selected from gamma radiation, ultraviolet radiation, or ozone exposure and incubated in an antibacterial and antifungal cocktail solution.

In certain embodiment, the processed tissue is incubated in a sucrose solution at a concentration ranging from about 5% to about 15%, such as 5.0%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, or ranges including and/or spanning the aforementioned values and a dextran solution at a concentration ranging from about 0.50% to about 1.50%, such as 0.50%, 0.55%, 0.60%, 0.65%, 0.70%, 0.75%, 0.80%, 0.85%, 0.90%, 0.95%, 1.0%, 1.05%, 1.10%, 1.15%, 1.20%, 1.25%, 1.30%, 1.35%, 1.40%, 1.45%, 1.50%, or ranges including and/or spanning the aforementioned values. The processed adipose tissue is incubated in antibacterial and antifungal cocktail solution at a temperature ranging from about −75.0° C. to about −85.0° C., such as −75.0° C., −76.0° C., −77.0° C., −78.0° C., −79.0° C., −80.0° C., −81.0° C., −82.0° C., −83.0° C., −84.0° C., −85.0° C., or ranges including and/or spanning the aforementioned values. The processed adipose tissue can be lyophilized followed by sterilization using irradiation from a group selected from gamma radiation, ultraviolet radiation, or ozone exposure.

In some embodiment, the sterilized adipose tissue can be packaged in a vacuum-sealed plastic container and stored as an allogeneic subcutaneous adipose tissue at a temperature ranging from about 2.0° C. to about 6.0° C., such as 2.0° C., 2.5° C., 3.0° C., 3.5° C., 4.0° C., 4.5° C., 5.0° C., 5.5° C., 6.0° C., or ranges including and/or spanning the aforementioned values for 6 months. The sterilized adipose tissue can also be packaged in a vacuum container (e.g., a plastic vacuum container) and stored as an allogeneic subcutaneous adipose tissue at a temperature ranging from about −15.0° C. to about −25.0° C., such as −15.0° C., −16.0° C., −17.0° C., −18.0° C., −19.0° C., −20.0° C., −21.0° C., −22.0° C., −23.0° C., −24.0° C., −25.0° C., or ranges including and/or spanning the aforementioned values for 12 months. In some embodiments, the allogeneic subcutaneous adipose tissue can be reconstituted in a saline solution or in a ringer solution before use.

In embodiments, the sterilized adipose tissue can be reconstituted in a saline solution and stored as an allogeneic subcutaneous adipose tissue at a temperature ranging from about −20.0° C. to about −80.0° C., such as −20.0° C., −25.0° C., −30.0° C., −35.0° C., −40.0° C., −45.0° C., −50.0° C., −55.0° C., −60.0° C., −65.0° C., −70.0° C., −75.0° C., −80.0° C., or ranges including and/or spanning the aforementioned values for no more than 12 months. The sterilized adipose tissue can also be reconstituted in a ringer solution and stored as an allogeneic subcutaneous adipose tissue at a temperature ranging from about −20.0° C. to about −80.0° C., such as −20.0° C., −25.0° C., −30.0° C., −35.0° C., −40.0° C., −45.0° C., −50.0° C., −55.0° C., −60.0° C., −65.0° C., −70.0° C., −75.0° C., −80.0° C., or ranges including and/or spanning the aforementioned values for no more than 12 months. The allogeneic subcutaneous adipose tissue can be reconstituted by thawing it before use.

In some embodiments, the allogeneic subcutaneous adipose tissue can be malleable and trimmable, and can be used for reconstruction surgeries and/or for cosmetic surgeries.

In certain embodiments, a method of preserving xenogeneic subcutaneous adipose tissue disclosed herein can include application of a set of treatments to an intact adipose tissue from a donor/cadaver of different species that may include receiving an adipose tissue, trimming vasculatures, skin, and fascia from the adipose tissue, incubating the adipose tissue in a buffer solution until freezing, thawing the frozen adipose tissue using a cryogenic solution, incubating the adipose tissue in a digestion solution, incubating the adipose tissue in a concentrated ionic solution, extracting polar cellular components from the adipose tissue, incubating the subcutaneous adipose tissue in a non-ionic surfactant solutions, washing the subcutaneous adipose tissue in ethanol or IPA or methanol, wherein the adipose tissue is processed, lyophilization of the adipose tissue, sterilizing the adipose tissue, and incubating the adipose tissue in an anti-inflammatory/antibiotics cocktail and sucrose and dextran solution.

In embodiments, the adipose tissue can be extracted from a different donor/cadaver or a donor/cadaver of a different species. In some embodiments, the adipose tissue extracted from a donor/cadaver can be processed as a fresh tissue. In some embodiments, the adipose tissue can be incubated in the buffer solution until freezing which can disrupt the phospholipid membrane and to expose the cytoplasm and nucleus. In some embodiments, the adipose tissue can be frozen at a temperature ranging from about −75.0° C. to about −85.0° C., such as −75.0° C., −76.0° C., −77.0° C., −78.0° C., −79.0° C., −80.0° C., −81.0° C., −82.0° C., −83.0° C., −84.0° C., −85.0° C., or ranges including and/or spanning the aforementioned values. In some embodiments, the buffer solution contains 25 mM HEPES. In some embodiments, the frozen adipose tissue is thawed in a cryogenic solution. In some embodiments, the frozen adipose tissue is thawed at a temperature ranging from about 36.0° C. to about 38.0° C., such as 36.0° C., 36.5° C., 37.0° C., 37.5° C., 38.0° C., or ranges including and/or spanning the aforementioned values. In some embodiments, the cryogenic solution can contain 1% or approximately 1% of a lipase inhibitor. The cryogenic solution can include an antibacterial and antifungal cocktail solution. In some embodiments, the lipase inhibitor can be Tetrahydrolipstatin. In some embodiments, the adipose tissue is incubated in a digestion solution with agitation at a temperature ranging from about 36.0° C. to about 38.0° C., such as 36.0° C., 36.5° C., 37.0° C., 37.5° C., 38.0° C., or ranges including and/or spanning the aforementioned values. In some embodiments, the adipose tissue is incubated in a digestion solution for a time ranging from about 6 hours to about 24 hours, such as 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or ranges including and/or spanning the aforementioned values. The digestion solution can contain EDTA-Trypsin solution at a concentration ranging from about 0.15% to about 0.35%, such as 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, or ranges including and/or spanning the aforementioned values. In some embodiments, the digestion solution can include an antibacterial and antifungal cocktail solution.

In some embodiments, the adipose tissue can be incubated in a concentrated ionic solution at a temperature ranging from about 36.0° C. to about 38.0° C., such as 36.0° C., 36.5° C., 37.0° C., 37.5° C., 38.0° C., or ranges including and/or spanning the aforementioned values. In some embodiments, the concentrated ionic solution contains antibacterial and antifungal cocktail solution. In some embodiments, the adipose tissue is incubated in a concentrated ionic solution for a time ranging from about 1 hours to about 3 hours, such as 1 hours, 1.5 hours, 2 hours, 2.5 hours, 3 hours, or ranges including and/or spanning the aforementioned values. In some embodiments, the concentrated ionic solution contains NaCl to extract the polar cellular component at a concentration ranging from about 0.5M to about 2M, such as 0.5M, 0.6M, 0.7M, 0.8M, 0.9M, 1.0M, 1.1M, 1.2M, 1.3M, 1.4M, 1.5M, 1.6M, 1.7M, 1.8M, 1.9M, 2.0M, or ranges including and/or spanning the aforementioned values. The concentrated ionic solution can contain 1% or approximately 1% of a lipase inhibitor.

In some embodiments, the adipose tissue can be incubated in a non-ionic surfactant to extract all the cellular component. The non-ionic surfactant can contain Triton X-100 solution at a concentration ranging from about 0.1% to about 1.0%, such as 0.1%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.0. %, or ranges including and/or spanning the aforementioned values. In some embodiments, the adipose tissue is incubated in the non-ionic surfactant for a time ranging from about 1 hours to about 3 hours, such as 1 hours, 1.5 hours, 2 hours, 2.5 hours, 3 hours, or ranges including and/or spanning the aforementioned values.

In some embodiments, the adipose tissue is washed in ethanol or IPA or methanol with agitation for a time ranging from about 1 hours to about 3 hours, such as 1 hours, 1.5 hours, 2 hours, 2.5 hours, 3 hours, or ranges including and/or spanning the aforementioned values. In some embodiments, the adipose tissue is washed in ethanol or IPA or methanol at a temperature ranging from about 2.0° C. to about 6.0° C., such as 2.0° C., 2.5° C., 3.0° C., 3.5° C., 4.0° C., 4.5° C., 5.0° C., 5.5° C., 6.0° C., or ranges including and/or spanning the aforementioned values.

In embodiments, the processed adipose tissue can be used fresh. The fresh processed adipose tissue can be sterilized using irradiation from a group selected from gamma radiation, ultraviolet radiation, or ozone exposure and incubated in antibacterial and antifungal cocktail solution.

In particular embodiments, the processed tissue is incubated in a sucrose solution at a concentration ranging from about 5% to about 15%, such as 5.0%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, or ranges including and/or spanning the aforementioned values and a dextran solution at a concentration ranging from about 0.50% to about 1.50%, such as 0.50%, 0.55%, 0.60%, 0.65%, 0.70%, 0.75%, 0.80%, 0.85%, 0.90%, 0.95%, 1.0%, 1.05%, 1.10%, 1.15%, 1.20%, 1.25%, 1.30%, 1.35%, 1.40%, 1.45%, 1.50%, or ranges including and/or spanning the aforementioned values. The processed adipose tissue is incubated in antibacterial and antifungal cocktail solution at a temperature ranging from about −75.0° C. to about −85.0° C., such as −75.0° C., −76.0° C., −77.0° C., −78.0° C., −79.0° C., −80.0° C., −81.0° C., −82.0° C., −83.0° C., −84.0° C., −85.0° C., or ranges including and/or spanning the aforementioned values. The processed adipose tissue can be lyophilized followed by sterilization using irradiation from a group selected from gamma radiation, ultraviolet radiation, or ozone exposure.

In some embodiments, the sterilized adipose tissue can be packaged in a vacuum-sealed plastic container and stored as an xenogeneic subcutaneous adipose tissue at a temperature ranging from about 2.0° C. to about 6.0° C., such as 2.0° C., 2.5° C., 3.0° C., 3.5° C., 4.0° C., 4.5° C., 5.0° C., 5.5° C., 6.0° C., or ranges including and/or spanning the aforementioned values for 6 months. The sterilized adipose tissue can also be packaged in a vacuum plastic container and stored as an xenogeneic subcutaneous adipose tissue at a temperature ranging from about −15.0° C. to about −25.0° C., such as −15.0° C., −16.0° C., −17.0° C., −18.0° C., −19.0° C., −20.0° C., −21.0° C., −22.0° C., −23.0° C., −24.0° C., −25.0° C., or ranges including and/or spanning the aforementioned values for 12 months. The xenogeneic subcutaneous adipose tissue can be reconstituted in a saline solution before use. The xenogeneic subcutaneous adipose tissue can also be reconstituted in a ringer solution before use.

In embodiments, the sterilized adipose tissue can be reconstituted in a saline solution and stored as an xenogeneic subcutaneous adipose tissue at a temperature ranging from about −20.0° C. to about −80.0° C., such as −20.0° C., −25.0° C., −30.0° C., −35.0° C., −40.0° C., −45.0° C., −50.0° C., −55.0° C., −60.0° C., −65.0° C., −70.0° C., −75.0° C., −80.0° C., or ranges including and/or spanning the aforementioned values for no more than 12 months. The sterilized adipose tissue can also be reconstituted in a ringer solution and stored as an xenogeneic subcutaneous adipose tissue at a temperature ranging from about −20.0° C. to about −80.0° C., such as −20.0° C., −25.0° C., −30.0° C., −35.0° C., −40.0° C., −45.0° C., −50.0° C., −55.0° C., −60.0° C., −65.0° C., −70.0° C., −75.0° C., −80.0° C., or ranges including and/or spanning the aforementioned values for no more than 12 months. The xenogeneic subcutaneous adipose tissue can be reconstituted by thawing it before use.

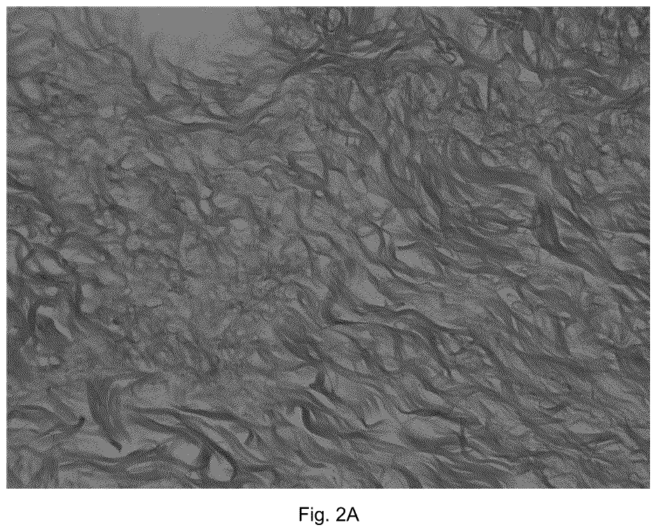

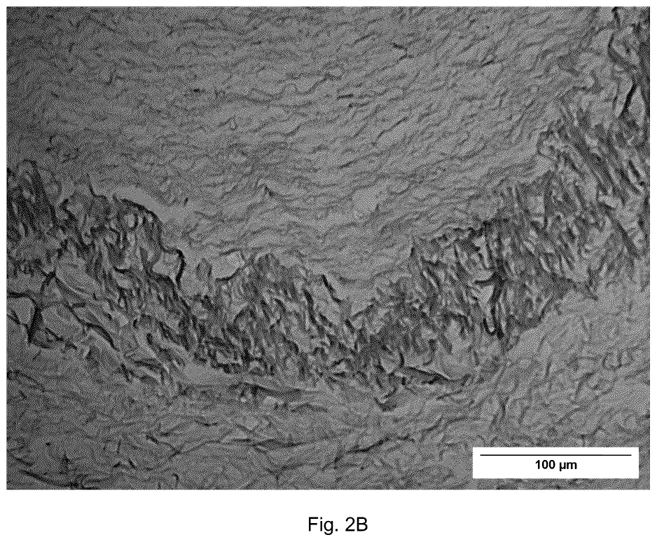

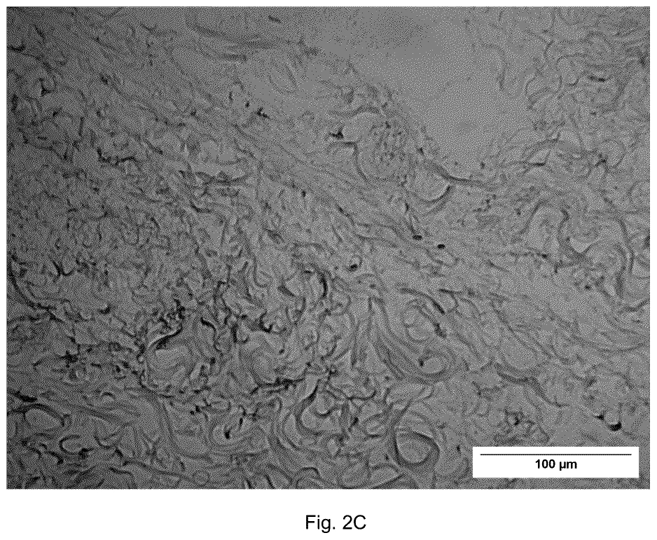

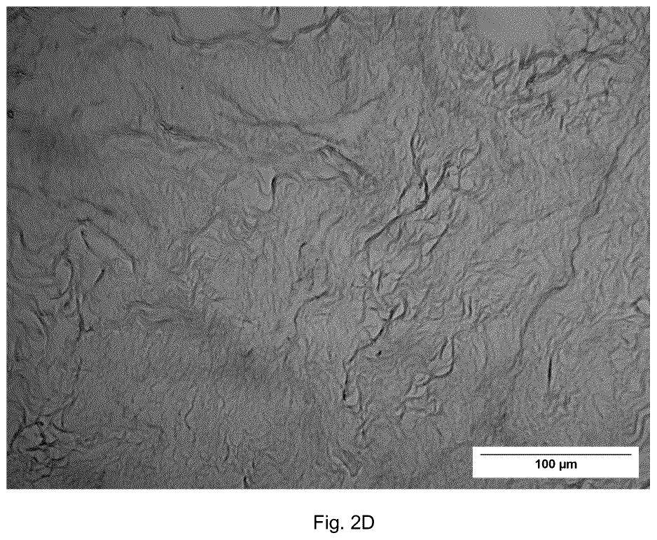

FIG. 2A-D shows microscopic images of the collagen fiber of a bovine adipose tissue where the extracellular matrix is intact and the cell bodies of the adipose tissue have been completely removed, using methods such as described herein.

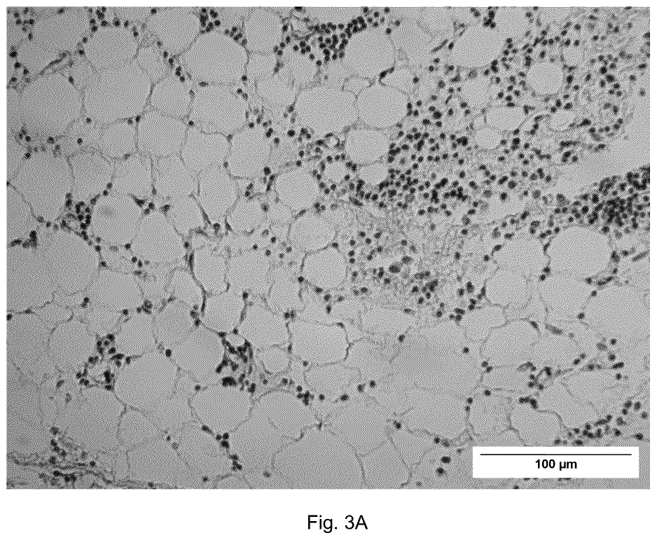

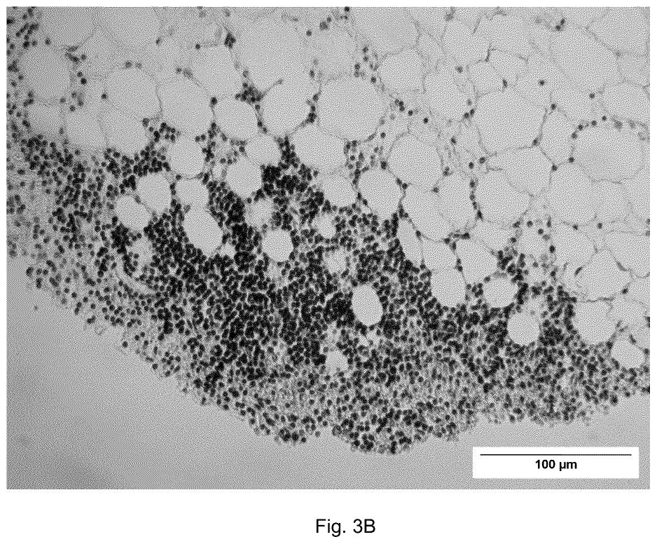

FIG. 3A-B shows microscopic images of the collagen fiber of a non-processed bovine adipose tissue. These images are used as controls. The images show that in the non-processed adipose tissue, both the web of extracellular matrix and the cell bodies of the adipose tissue are intact.

By comparing the images from FIGS. 2 and 3 shows the benefit of using the methods described in the present disclosure to successfully remove any cellular bodies from the processed adipose tissue thereby minimizing potential immunogenic reaction after transplantation while maintaining the general cytoarchitecture of the adipose tissue. In addition, the method may prevent any additional anesthesia time, pain and discomfort for the patient. In addition, the patient may not have to take extra care for the area from which the fat has been grafted and avoid infection and other injury to the patient on the graft site. The method described in the present disclosure may also prevent any permanent scarring or other skin defects at the graft site for the patient.

As used herein and unless otherwise stated or implied by context, terms that are used herein have the meanings that are defined here. The descriptions of embodiments and examples that are described illustrate the disclosure and they are not intended to limit it in any way. Unless otherwise contraindicated or implied, e.g., by including mutually exclusive elements or options, in these definitions and throughout this specification, the terms “a” and “an” mean one or more and the term “or” means and/or.

As used herein, “allogeneic” means, or otherwise refers to, one or more cells or tissues that are genetically and immunologically different because they are derived from different species.

As used herein, “xenogeneic” means, or otherwise refers to, one or more cells or tissues that are genetically different because they are derived from separate individuals of the same species that are sufficiently unlike genetically to interact antigenically.

As used herein, “adipose tissue” means, or otherwise refers to, fat or a loose connective tissue that extends throughout the body underneath the skin, between your internal organs, and in the inner cavities of bones. This tissue consists of lipid-rich cells called adipocytes.

As used herein, “subcutaneous” means, or otherwise refers to being, living, occurring, or administered under the skin.

As used herein, “preservation” “preserving,” and “preserve” have their ordinary and customary meaning in the art. These terms generally refer to an act, process or result of protecting something from being decayed or damaged. The terms “preservation,” as used herein shall be given its ordinary meaning and shall also cover any preservation of cells or tissues of any mammal, such as a human, and includes: (a) conserving cells or tissues before it is used; and/or (b) maintaining the structure and function of the cells or tissues.

As used herein, “sterilization” and “sterilize” have their ordinary and customary meaning in the art. These terms generally refer to a process of removing, killing or deactivating any pathogens or living microorganisms. The process of sterilization takes place by treating something with chemicals or subjecting it to high heat or radiation.

As used herein, “donor/cadaver” means, or otherwise refers to, someone who gives part of their body such as cells, tissues, organs, or their blood for transplantation.

All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the general description and the following detailed description are exemplary and explanatory only and are not restrictive. The term “and/or” denotes that the provided possibilities can be used together or be used in the alternative. Thus, the term “and/or” denotes that both options exist for that set of possibilities.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the disclosure. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

The foregoing description provides context and examples but should not be interpreted to limit the scope of the disclosure covered by the claims that follow in this specification or in any other application that claims priority to this specification. No single component or collection of components is essential or indispensable. For example, in some embodiments one or more variables, such as Y or Y and Q may be omitted. Any feature, structure, component, material, step, or method that is described and/or illustrated in any embodiment in this specification can be used with or instead of any feature, structure, component, material, step, or method that is described and/or illustrated in any other embodiment in this specification.

While certain arrangements of the inventions have been described, these arrangements have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, arrangement, or example are to be understood to be applicable to any other aspect, arrangement or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing arrangements. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some arrangements, the actual steps taken in the processes illustrated and/or disclosed may differ from those shown in the figures. Depending on the arrangement, certain of the steps described above may be removed, others may be added. Furthermore, the features and attributes of the specific arrangements disclosed above may be combined in different ways to form additional arrangements, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular arrangement. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain arrangements include, while other arrangements do not include, certain features, elements, and/or steps. Thus, such conditional language is not generally intended to imply that features, elements, and/or steps are in any way required for one or more arrangements or that one or more arrangements necessarily include logic for deciding, with or without user input or prompting, whether these features, elements, and/or steps are included or are to be performed in any particular arrangement.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain arrangements require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain arrangements, the terms “generally parallel” and “substantially parallel” refer to a value, amount, or characteristic that departs from exactly parallel by less than or equal to 15°, 10°, 5°, 3°, 1 degree, or 0.1 degree. The ranges disclosed herein also encompass any and all overlap, sub-ranges, and combinations thereof, and any specific values within those ranges. Language such as “up to,” “at least,” “greater than,” “less than,” “between,” and the like includes the number recited. Numbers and values used herein preceded by a term such as “about” or “approximately” include the recited numbers. For example, “approximately 7 mm” includes “7 mm” and numbers and ranges preceded by a term such as “about” or “approximately” should be interpreted as disclosing numbers and ranges with or without such a term in front of the number or value such that this application supports claiming the numbers, values and ranges disclosed in the specification and/or claims with or without the term such as “about” or “approximately” before such numbers, values or ranges such, for example, that “approximately two times to approximately five times” also includes the disclosure of the range of “two times to five times.” The scope of the present disclosure is not intended to be limited by the specific disclosures of preferred arrangements in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A processed adipose tissue, comprising:
an adipose tissue extracted from a human or animal donor, the adipose tissue processed such that cells are removed from the tissue; and
wherein the adipose tissue contains collagen, elastin, fibrin, lipids and extracellular matrix.

2. The processed adipose tissue of claim 1, wherein the adipose tissue is allogeneic and/or is xenogeneic.

3. (canceled)

4. (canceled)

5. (canceled)

6. The processed adipose tissue of claim 1, wherein the adipose tissue is stored in a concentrated ionic solution to remove polar cellular components.

7. The processed adipose tissue of claim 6, wherein the concentrated ionic solution comprises lipase inhibitor.

8. (canceled)

9. (canceled)

10. The processed adipose tissue of claim 1, wherein the adipose tissue is lyophilized using sucrose and dextran.

11. The processed adipose tissue of claim 1, wherein the adipose tissue is sterilized using gamma radiation, ultraviolet radiation or ozone exposure.

12. The processed adipose tissue of claim 11, wherein the sterilized adipose tissue is configured to be stored in a vacuum-sealed plastic container for up to 12 months.

13. A method of processing a subcutaneous adipose tissue comprising application of a set of treatments to an intact adipose tissue from a donor/cadaver, the set of treatments comprising:
receiving an adipose tissue;
trimming vasculatures, skin, and fascia from the adipose tissue;
incubating the adipose tissue in a buffer solution;
thawing the frozen adipose tissue using a cryogenic solution;
incubating the adipose tissue in a digestion solution;
incubating the adipose tissue in a concentrated ionic solution overnight;
extracting polar cellular components from the adipose tissue;
incubating the adipose tissue in a non-ionic surfactant solutions; and
washing the subcutaneous adipose tissue in ethanol or IPA or methanol;
whereby the subcutaneous adipose tissue is made.

14. The method of claim 13, wherein the adipose tissue is allogeneic or xenogeneic.

15. (canceled)

16. (canceled)

17. The method of claim 13, wherein the buffer solution comprises 25 mM HEPES.

18. The method of claim 13, wherein the cryogenic solution comprises 1% lipase inhibitor.

19. The method of claim 13, wherein the digestion solution comprises 0.25% EDTA-trypsin solution.

20. (canceled)

21. The method of claim 13, wherein the concentrated ionic solution comprises 1M NaCl and/or 1% lipase inhibitor.

22. (canceled)

23. (canceled)

24. (canceled)

25. (canceled)

26. (canceled)

27. (canceled)

28. (canceled)

29. (canceled)

30. A method of preserving a subcutaneous adipose tissue for cosmetic and reconstruction surgeries comprising:
receiving an adipose tissue;
trimming vasculatures, skin, and fascia from the adipose tissue;
incubating the adipose tissue in a buffer solution until freezing;
thawing the frozen adipose tissue using a cryogenic solution;
incubating the adipose tissue in a digestion solution;
incubating the adipose tissue in a concentrated ionic solution;
extracting polar cellular components from the adipose tissue;
incubating the subcutaneous adipose tissue in a non-ionic surfactant solutions;
washing the subcutaneous adipose tissue in ethanol or IPA or methanol; wherein the adipose tissue is processed;
sterilizing the processed adipose tissue;
incubating the adipose tissue in an anti-inflammatory/antibiotics cocktail and sucrose and dextran solution;
packaging the sterilized adipose tissue in a vacuum-sealed plastic container; and
storing the sterilized adipose tissue.

31. The method of claim 30, further comprising lyophilization of the processed adipose tissue.

32. The method of claim 30, wherein the buffer solution comprises 25 mM HEPES.

33. The method of claim 30, wherein the cryogenic solution comprises 1% lipase inhibitor.

34. The method of claim 30, wherein the digestion solution comprises 0.25% EDTA-trypsin solution.

35. (canceled)

36. The method of claim 30, wherein the concentrated ionic solution comprises 1M NaCl and/or 1% lipase inhibitor.

37. (canceled)

38. (canceled)

39. (canceled)

40. (canceled)

41. (canceled)

42. (canceled)

43. (canceled)

44. The method of claim 30, further comprising storing the sterilized adipose tissue at 4° C. for up to 6 months, or at −20° C. for up to 12 months.

45. (canceled)

46. (canceled)

47. (canceled)

48. (canceled)

49. (canceled)
